(19) United States
(12) Patent Application Publication
(10) Pub. No.: US 2005/0119549 A1
(43) Pub. Date: Jun. 2, 2005

(54) Embedded metal-programmable image processing array for digital still camera and camrecorder products

(76) Inventors: Anastassios Markas, Cary, NC (US); Clifford S. Hofman, Cary, NC (US); Jason P. Hurst, Raleigh, NC (US); Michael G. McNamer, Cary, NC (US)

Correspondence Address: SAWYER LAW GROUP LLP, P.O. Box 51418, Palo Alto, CA, 94303, US

(21) Appl. No.: 10/960,354

(22) Filed: Oct. 6, 2004

Related U.S. Application Data
(60) Provisional application No. 60/525,932, filed on Dec. 1, 2003.

(51) Int. Cl. A61B 5/05

(52) U.S. Cl. 600/407

(57) ABSTRACT

A method and system for providing an application specific integrated circuit (ASIC) for a digital image processing system is disclosed. The method and system include providing a microprocessor subsystem and providing an image processing subsystem. The microprocessor subsystem controls the digital image processing system. The image processing subsystem includes image processing hardware and programmable logic. The programmable logic includes a plurality of programmable cells that are customized during fabrication of the ASIC.

### CROSS-REFERENCE TO RELATED APPLICATION

This application is claiming under 35 USC 119(e) the benefit of provisional patent application Ser. No. 60/525,932 filed on Dec. 1, 2003.

### FIELD OF THE INVENTION

The present invention relates to image processing subsystems, and more particularly to a method and system for providing an embedded programmable image processing array for digital imaging devices such as digital still cameras and digital camrecorders.

### BACKGROUND OF THE INVENTION

Digital imaging devices, such as digital still cameras and digital camrecorders, employ application specific integrated circuits (ASICs) capable of controlling the digital imaging device and performing image processing. FIG. 1 depicts a conventional ASIC 10 used in a digital imaging device. The conventional ASIC 10 is partitioned into two subsystems, a conventional image processing subsystem 20 and a conventional microprocessor subsystem 40. The conventional microprocessor subsystem 40 includes a processor 42, such as an ARM processor 42. The conventional microprocessor subsystem 40 controls the digital imaging device (not shown) in which the ASIC is used. The conventional microprocessor subsystem 40 also includes serial interfaces 44, flash card interfaces 46, memory 48, direct memory access (DMA) unit 50, analog interfaces 52, timers 54, interrupt controller 56, RAM 58, realtime clock 60, watchdog timer 62, register files 64, cache controller 66, and prog/data cache 68. The conventional image processing subsystem 20 includes a SDRAM interface 22, a pixel interface 24, an evaluation block 26, a conventional image processing core or digital signal processor (DSP) 28, JPEG and MPEG image coder/decoders 30 and 32, respectively, display interfaces 34, and JPEG/SDRAM interface 36.

The conventional image processing subsystem 20 typically includes either the conventional image processing core or DSP 28. The conventional image processing core 28 is fast, capable of rapidly implementing image processing algorithms. The conventional image processing core 28 also consumes less power. If the conventional image processing subsystem 20 utilizes a DSP 28, then the conventional image processing subsystem 20 has a high degree of programmability.

Although the conventional image processing subsystem 20 functions, one of ordinary skill in the art will readily recognize that there are drawbacks to use of either the conventional image processing core or the DSP 28. Although the DSP 28 is programmable, it is subject to high power consumption and low speed of execution. Similarly, although the conventional image processing core 28 consumes less power and is faster, it is not programmable. Furthermore, the conventional image processing subsystem is not easily customizable. Therefore, it may be difficult for a maker of digital imaging devices to obtain and ASIC that implements intellectual property proprietary to the maker or to accommodate changes in technology.

Accordingly, what is needed is a system and method for providing an improved ASIC having greater flexibility without sacrificing performance. The present invention addresses such a need.

### SUMMARY OF THE INVENTION

The present invention provides a method and system for providing an application specific integrated circuit (ASIC) for a digital image processing system. The method and system comprise providing a microprocessor subsystem and providing an image processing subsystem. The microprocessor subsystem controls the digital image processing system. The image processing subsystem includes image processing hardware and programmable logic. The programmable logic includes a plurality of programmable cells that are customized during fabrication of the ASIC.

According to the system and method disclosed herein, the present invention provides an ASIC for digital image processing systems that is easily and rapidly customizable during fabrication yet provides the speed and cost benefits of hardware.

### BRIEF DESCRIPTION OF THE DRAWINGS

FIG. 1 is a diagram of a conventional ASIC for a digital imaging device.

FIG. 2A is a high-level block diagram of one embodiment of an ASIC in accordance with the present invention having an embedded programmable logic for use in a digital imaging device.

FIG. 2B is a more detailed block diagram of one embodiment of an ASIC in accordance with the present invention having an embedded programmable logic for use in a digital imaging device.

FIG. 3 is a diagram of the operation of one embodiment of an ASIC in accordance with the present invention during image processing system.

FIG. 4 is a high-level flow chart depicting one embodiment of a method in accordance with the present invention for providing an ASIC used in digital imaging devices and having an embedded programmable logic.

FIG. 5 is a more detailed flow chart depicting one embodiment of a method in accordance with the present invention for providing an ASIC used in digital imaging devices and having an embedded programmable logic.

FIG. 6 is a more detailed flow chart depicting another embodiment of a method in accordance with the present invention for providing an ASIC used in digital imaging devices and having an embedded programmable logic.

### DETAILED DESCRIPTION OF THE INVENTION

The present invention relates to an improvement in ASICs. The following description is presented to enable one of ordinary skill in the art to make and use the invention and is provided in the context of a patent application and its requirements. Various modifications to the preferred embodiment will be readily apparent to those skilled in the art and the generic principles herein may be applied to other embodiments. Thus, the present invention is not intended to be limited to the embodiment shown, but is to be accorded the widest scope consistent with the principles and features described herein.

The present invention provides a method and system for providing an application specific integrated circuit (ASIC) for a digital image processing system. The method and system comprise providing a microprocessor subsystem and providing an image processing subsystem. The microprocessor subsystem controls the digital image processing system. The image processing subsystem includes image processing hardware and a programmable logic. The programmable logic includes a plurality of programmable cells that are customized during fabrication of the ASIC.

The present invention will be described in terms of particular functions being performed by the programmable logic. However, one of ordinary skill in the art will readily recognize that this method and system will operate effectively for other functions being performed by the programmable logic. The present invention is also described in the context of particular components and methods including certain steps. However, one of ordinary skill in the art will readily recognize that the method and system operate effectively for other components and methods having steps not incompatible with the method and system described herein.

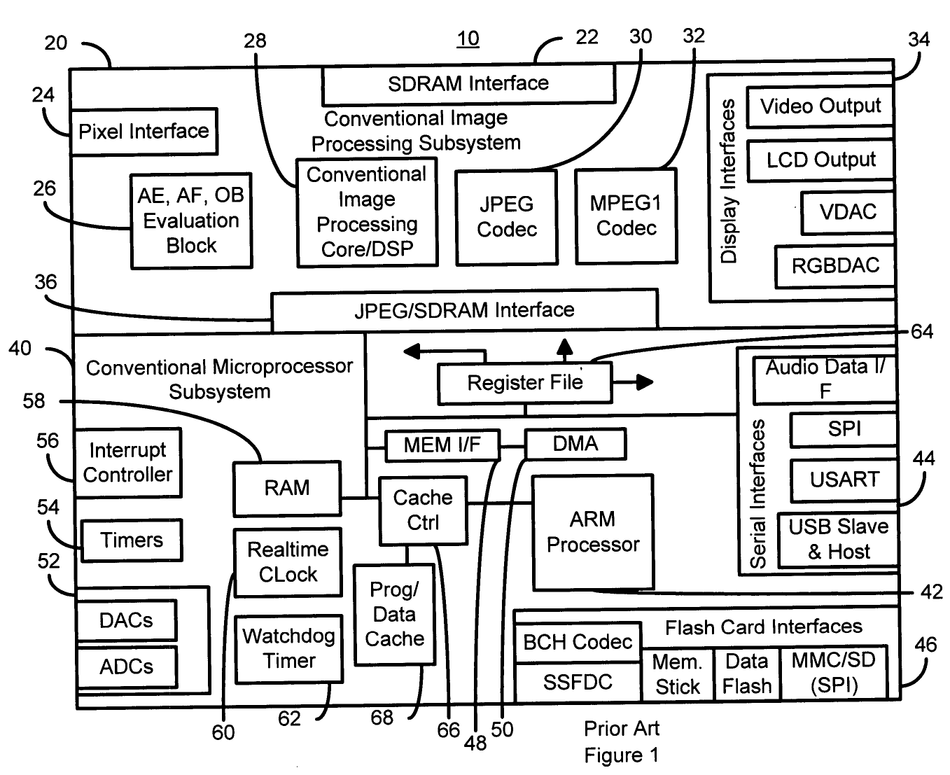

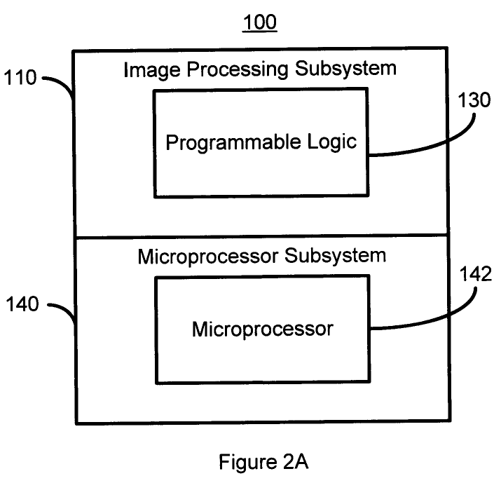

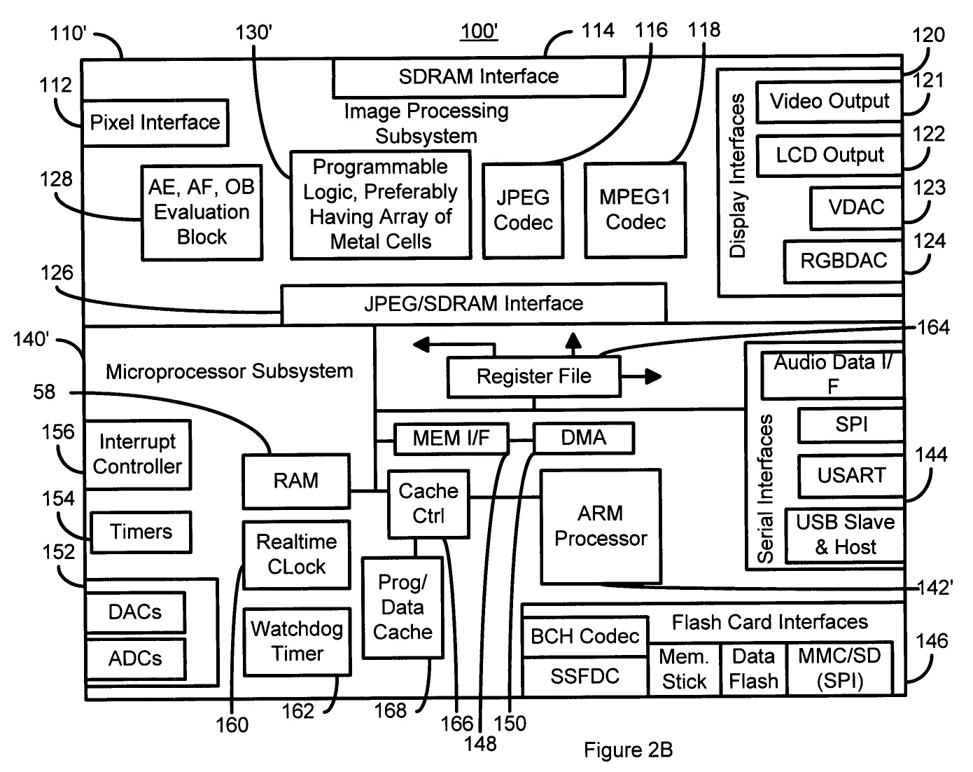

To more particularly illustrate the method and system in accordance with the present invention, refer now to FIG. 2A, depicting a high-level block diagram of one embodiment of an ASIC 100 in accordance with the present invention for use in a digital imaging device, such as a digital still camera or digital camrecorder. The ASIC 100 includes an image processing subsystem 110 and a microprocessor subsystem 140. The microprocessor subsystem 140 includes a microprocessor 142. In a preferred embodiment, the microprocessor 142 is an ARM processor. The microprocessor subsystem 140 may be analogous to the conventional microprocessor subsystem 40. The image processing subsystem 110 includes programmable logic 130, as well as image processing hardware (not explicitly depicted in FIG. 2A). As used herein, the programmable logic 130 is logic that is rapidly and easily customized during fabrication. The logic 130 is more easily customizable than conventional logic, such as the conventional logic 28 depicted in FIG. 1. Referring back to FIG. 2A, the programmable logic 130 includes an array of cells that can be customized during fabrication. The programmable logic 130 preferably includes an array of programmable metal cells. Such an array of metal cells preferably has the underlying gates fabricated to set specifications. However, the metal layers connecting the gates may be rapidly and easily customized by altering the metal masks used in fabricating the programmable logic 130. The functions provided by the underlying gates are rapidly and easily changed. Thus, the metal cells may be rapidly and relatively easily programmed during fabrication. Consequently, the programmable logic 130 may be customized during fabrication. In a preferred embodiment, the metal cells are customized by altering the metal masks used in fabricating the programmable logic 130.

The programmable logic 130 is customizable and offers some degree of programmability. Thus, the flexibility of the ASIC 100 is improved. Because the programmable logic 130 is hardware based, the cost and power consumption of the programmable logic 130 is relatively low. For the same reasons, the speed of the programmable logic 130 is relatively high. Consequently, the benefits of both the conventional image processing core and DSP 28 can be achieved substantially without the drawbacks of either. Moreover, the programmable logic 130 provides a common platform for software development for different digital imaging device makers. As a result, the ASIC 100 can be relatively quickly and easily customized for different makers of digital imaging devices while allowing the makers to maintain the same system software.

FIG. 2B is a more detailed block diagram of one embodiment of an ASIC 100′ in accordance with the present invention having an embedded programmable logic for use in a digital imaging device. Components of the ASIC 100′ correspond to the ASIC 100 depicted in FIG. 2A. Consequently, the ASIC 100′ includes an image processing subsystem 110′ and a microprocessor subsystem 140′. The image processing subsystem 110′ includes programmable logic 130′ including an array of metal cells. Thus, the programmable logic 130′ is analogous to the programmable logic 130 depicted in FIG. 2A. Similarly, the microprocessor subsystem 140′ includes a microprocessor 142′ that is preferably an ARM processor. Thus, the microprocessor 142′ corresponds to the microprocessor 142 depicted in FIG. 2A. The conventional microprocessor subsystem 140 also includes serial interfaces 144, flash card interfaces 146, memory 148, DMA unit 150, analog interfaces 152, timers 154, interrupt controllers 156, RAM 158, realtime clock 160, watchdog timer 162, register files 164, cache controller 166, and program/data cache 168.

In addition to the programmable logic 130′, the image processing subsystem 110′ includes hardware used in processing the image and performing analogous functions. In the embodiment shown, the image processing subsystem 110′ includes display interfaces 120 such as a video output 121, an LCD output 122, VDAC 123 and RGBDAC 124. The image processing subsystem 110′ shown also includes pixel interface 112, SDRAM interface 114, JPEG codec 116 and MPEG1 codec 118, JPEG/SDRAM interface 126, and AE, AF, OB evaluation block 128. The hardware elements 112, 114, 116, 118, 120, 121, 122, 123, 124, and 126 of the image processing subsystem 110′ are analogous to portions of the conventional image processing array 20 depicted in FIG. 1. Referring back to FIG. 2B, the programmable logic 130′ includes cells that may be rapidly and easily customized during fabrication. In a preferred embodiment, the cells are metal cells are customized by altering the metal mask used in fabricating the programmable logic 130. The tasks that may be customized using the programmable logic 130 include but are not limited to color recovery, noise filtering, image enhancement, gain control, color mapping, hue and saturation control, vignetting, shading lens correction, and other functions desired and defined by the customer for whom the ASIC is provided. In a preferred embodiment, the blocks described above, including video and image coding (blocks 116 and 118), image capture, image rotation, image scaling, video encoder and display processes, and the microprocessor subsystem.

The programmable logic 130′ is relatively easily customizable and thus can be programmed. Thus, the flexibility of the ASIC 100′ is improved. Because the programmable logic 130′ is hardware based, the cost and power consumption of the programmable logic 130′ is relatively low. For the same reasons, the speed of the programmable logic 130′ is relatively high. Consequently, the benefits of both the conventional image processing core and DSP 28 can be achieved substantially without the drawbacks of either. Moreover, the programmable logic 130′ provides a common platform for software development for different digital imaging device makers. As a result, the ASIC 100′ can be relatively quickly and easily customized for different makers of digital imaging devices while allowing the makers to develop some software for the programmable logic.

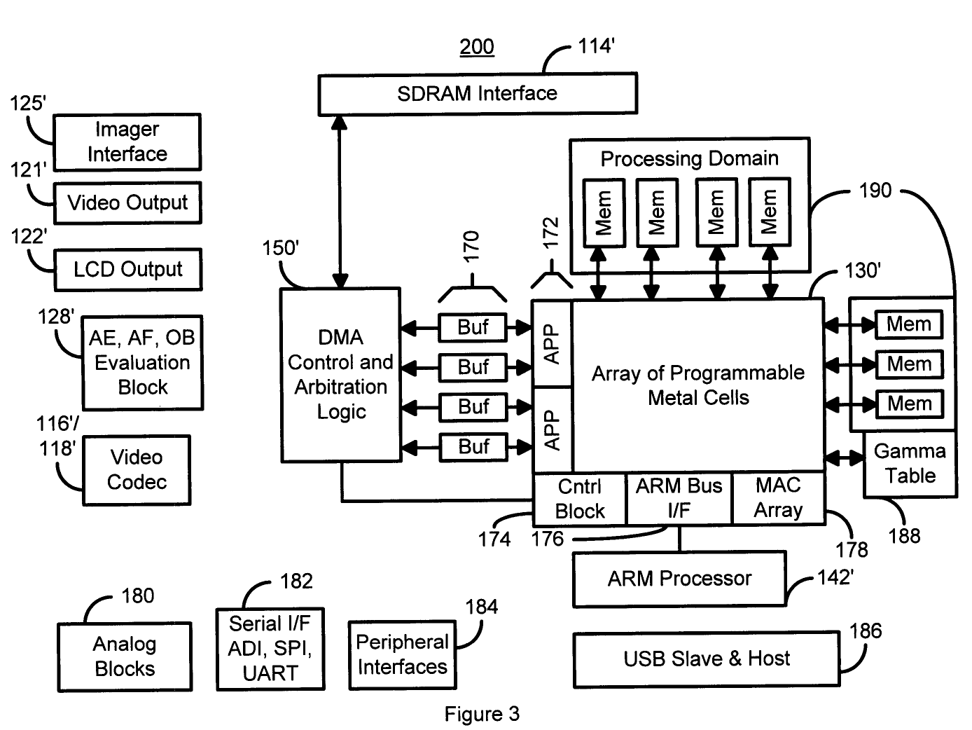

FIG. 3 is a diagram 200 of the operation of one embodiment of the ASIC 100′ during image processing system. Thus, portions of the ASIC 100′ are depicted. In addition, other portions of the ASIC 100′ not previously shown are depicted. Consequently, the diagram 200 includes buffers 170, APP logic blocks 172, control block 174, ARM bus I/F 176, MAC array 178, analog blocks 180, Serial I/F 182, peripheral interfaces 184, USB slave and host interface 186, gamma table 188, and memories 190 that has been split into two sections. During image processing, the programmable logic 130′ can achieve fixed input/output with the memories 190, ARM standard bus 176, DMA block 150, and control interface 174. For the programmable logic 130′, the memories 190 can be addressed as a single block or in multiple blocks to allow for a pipelined architecture. The ARM 142′, which is part of the processor block, can utilize the programmable logic 130′ through a customer-defined register file (not explicitly shown in FIG. 3) that accessed via the ARM bus 176. Moreover, a dedicated multiply accumulate array can be provided in the programmable logic 130′. The interface between the programmable logic 130′ and the DMA control and arbitration block 150′ can be used to control transfer to or from the SDRAM (not shown in FIG. 3). The APP logic block 170 can handle automatic ping-ponging to or from the memories 190, as well as provide coordinate information for the programmable logic 130′.

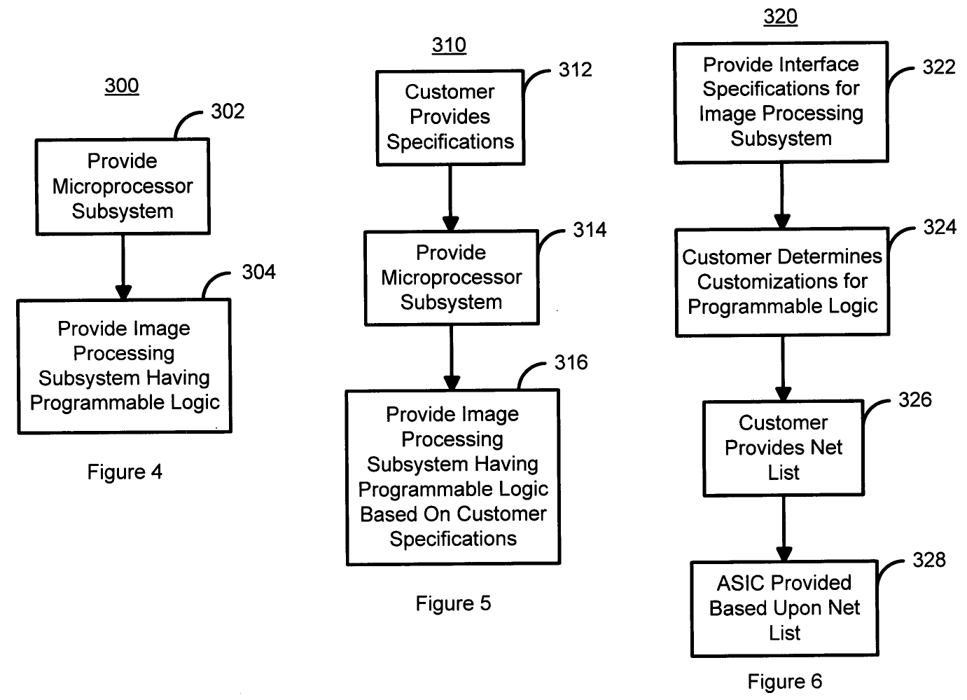

FIG. 4 is a high-level flow chart depicting one embodiment of a method 300 in accordance with the present invention for providing an ASIC used in digital imaging devices and having embedded programmable logic. The method 300 is described in the context of the ASIC 100. However, nothing prevents the method 300 being used with another ASIC, such as the ASIC 100′. The microprocessor subsystem 140 having a microprocessor 142 is provided as part of the ASIC 100, via step 302. The image processing subsystem 110 including the programmable logic 130 is provided, via step 304. Step 304 includes customizing the programmable logic 130, for example using metal masks for the metal cells contained in the programmable logic 130. Using the method 300, the ASIC 100 can be provided.

FIG. 5 is a more detailed flow chart depicting one embodiment of a method 310 in accordance with the present invention for providing an ASIC used in digital imaging devices and having an embedded programmable logic. The method 310 is described in the context of the ASIC 100. However, nothing prevents the method 310 being used with another ASIC, such as the ASIC 100′. A customer, particularly the maker of the digital imaging device, provides specifications for the programmable logic 130 to the maker of the ASIC, via step 312. The microprocessor subsystem 140 having a microprocessor 142 is provided as part of the ASIC 100, via step 314. The image processing subsystem 110 including the programmable logic 130 is provided, via step 316. Step 316 includes customizing the programmable logic 130 based upon the specifications provided by the customer. In a preferred embodiment, the programmable logic 130 is customized in step 316 by altering the metal masks used in fabricating the metal cells contained in the programmable logic. Using the method 300, the ASIC 100 can be provided and the benefits of the ASIC 100 achieved. Stated differently, using the method 310 a manufacturer can rapidly and easily respond to different customer's specifications and provide customized ASICs meeting these specifications.

FIG. 6 is a more detailed flow chart depicting another embodiment of a method 320 in accordance with the present invention for providing an ASIC used in digital imaging devices and having an embedded programmable logic. The method 320 is described in the context of the ASIC 100. However, nothing prevents the method 3210 being used with another ASIC, such as the ASIC 100′. The customer, generally a digital imaging device manufacturer, is provided with the specifications for the interface to the image processing subsystem 110, via step 322. The maker of the ASIC 100 thus informs the customer of how the programmable logic 130 may be customized. These specifications make it possible for the customer to determine how to tailor the ASIC 100 for the customer's product without disclosing the same proprietary information to the maker of the ASIC 100. The customer determines how to customize the programmable logic 130 for the customer's system, via step 324. The customer provides a net list for the ASIC 100 manufacturer, via step 326. In one embodiment, the net list can be for the programmable logic 130 alone. The net list determines how the gates of the programmable logic 130 are to be fabricated. More specifically, the net list informs the ASIC manufacturer of how the gates of the programmable logic 130 are to be routed, or coupled together. In the preferred embodiment, in which the programmable logic 130 includes metal cells, the net list determines the metal masks used in customizing the programmable logic 130. The ASIC 100 is manufactured based on the net list provided by the customer, via step 328.

Using the method 320, the ASIC 100 or 100′ can be manufactured. As a result, a manufacturer can rapidly and easily respond to different customer's needs and provide customized ASICs meeting these needs. Furthermore, because the customer determines how the programmable logic 130 is customized, customer need not disclose as much propriety information. Consequently, the method 320 provides the customer with additional security.

A method and system has been disclosed for providing an ASIC having an embedded programmable logic. Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

1. An application specific integrated circuit (ASIC) for a digital image processing system comprising:
a microprocessor subsystem for controlling the digital image processing system; and
an image processing subsystem including image processing hardware and programmable logic, the programmable logic including a plurality of programmable cells customizable during fabrication.

2. The ASIC of claim 1 wherein the microprocessor subsystem further includes an ARM processor.

3. The ASIC of claim 1 wherein the image processing hardware of the image processing subsystem includes video image/coding hardware.

4. The ASIC of claim 1 wherein the image processing hardware of the image processing subsystem includes at least one image coding block.

5. The ASIC of claim 1 wherein the plurality of programmable cells further include a plurality of metal cells configured during processing for use in color recovery.

6. The ASIC of claim 1 wherein the plurality of programmable cells further include a plurality of metal cells configured during processing for use in noise filtering.

7. The ASIC of claim 1 wherein the plurality of programmable cells further include a plurality of metal cells configured during processing for use in image enhancement.

8. The ASIC of claim 1 wherein the plurality of programmable cells further include a plurality of metal cells configured during processing for use in gain control.

9. The ASIC of claim 1 wherein the plurality of programmable cells further include a plurality of metal cells configured during processing for use in color mapping.

10. The ASIC of claim 1 wherein the plurality of programmable cells further include a plurality of metal cells configured during processing for use in hue/saturation control.

11. The ASIC of claim 1 wherein the plurality of programmable cells further include a plurality of metal cells configured during processing for use in vignetting and/or shading lens correction.

12. A method for providing an application specific integrated circuit (ASIC) for use in a digital image processing system comprising:
(a) providing a microprocessor subsystem for controlling the digital image processing system; and
(b) providing an image processing subsystem including image processing hardware and programmable logic, the programmable logic including a plurality of programmable cells customized during fabrication of the ASIC.

13. The method of claim 12 wherein the microprocessor subsystem providing step (a) further includes the step of:
(a1) utilizing an ARM processor.

14. The method of claim 12 wherein the image processing subsystem providing step (b) further includes the step of:
(b1) providing video image/coding hardware as part of the image processing hardware of the image processing subsystem.

15. The method of claim 1 wherein the image processing subsystem providing step (b) further includes the step of:
(b1) providing at least one image coding block as part of the image processing hardware of the image processing subsystem.

16. The method of claim 12 wherein the plurality of programmable cells further include a plurality of metal cells and wherein the image processing subsystem providing step (b) further includes the step of:
(b1) configuring the plurality of metal cells for use in color recovery.

17. The method of claim 12 wherein the plurality of programmable cells further include a plurality of metal cells and wherein the image processing subsystem providing step (b) further includes the step of:
(b1) configuring the plurality of metal cells for use in noise filtering.

18. The method of claim 12 wherein the plurality of programmable cells further include a plurality of metal cells and wherein the image processing subsystem providing step (b) further includes the step of:
(b1) configuring the plurality of metal cells for use in image enhancement.

19. The method of claim 12 wherein the plurality of programmable cells further include a plurality of metal cells and wherein the image processing subsystem providing step (b) further includes the step of:
(b1) configuring the plurality of metal cells for use in gain control.

20. The method of claim 12 wherein the plurality of programmable cells further include a plurality of metal cells and wherein the image processing subsystem providing step (b) further includes the step of:
(b1) configuring the plurality of metal cells for use in color mapping.

21. The method of claim 12 wherein the plurality of programmable cells further include a plurality of metal cells and wherein the image processing subsystem providing step (b) further includes the step of:
(b1) configuring the plurality of metal cells for use in hue/saturation control.

22. The method of claim 12 wherein the plurality of programmable cells further include a plurality of metal cells and wherein the image processing subsystem providing step (b) further includes the step of:
(b1) configuring the plurality of metal cells for use in vignetting and/or shading lens correction.

23. The method of claim 12 further comprising the steps of:
(c) receiving from a customer a plurality of specifications for the programmable logic.

24. The method of claim 23 wherein the image processing subsystem providing step (b) further includes the step of:
(b1) customizing the programmable logic during fabrication based upon the plurality of specifications.

25. The method of claim 24 wherein the plurality of programmable cells further include a plurality of metal cells and wherein the customizing step (b1) further includes the step of:
(b1i) customizing at least one metal mask based upon a portion of the plurality of specifications; and
(b1ii) fabricating the plurality of programmable metal cells using the at least one mask.

26. The method of claim 12 further comprising:
(c) providing the customer with a plurality of specifications for the programmable logic;
(d) allowing the customer to determine how the programmable logic is to be customized; and
(e) receiving from a customer a net list for at least the programmable logic.

27. The method of claim 26 wherein the image subsystem providing step (b) further includes:
(b1) providing the image processing subsystem based on the net list.
